(19) United States
(12) Patent Application Publication
(10) Pub. No.: US 2024/0108234 A1
(43) Pub. Date: Apr. 4, 2024

(54) BLOOD FLOW ASSESSMENT DEVICE, METHOD, AND PROGRAM

(71) Applicant: SHISEIDO COMPANY, LTD., Tokyo (JP)

(72) Inventors: Daisuke KAMIWANO, Tokyo (JP); Motoki TAKATA, Tokyo (JP); Megumi SEKINO, Tokyo (JP); Yohei KOBAYASHI, Tokyo (JP); Juncheng LI, Tokyo (JP); Naomi KANNO, Tokyo (JP)

(21) Appl. No.: 18/256,102

(22) PCT Filed: Dec. 24, 2021
(86) PCT No.: PCT/JP2021/048440

§ 371 Date: Jun. 6, 2023

(30) Foreign Application Priority Data
Jan. 6, 2021 (JP) 2021-001054

(51) Int. Cl. A61B 5/026; A61B 5/00; A61B 5/01

(52) U.S. Cl. CPC A61B 5/026; A61B 5/01; A61B 5/7271

(57) ABSTRACT

The present disclosure improves the accuracy of blood flow assessment. According to one embodiment of the present invention, a blood flow assessment device has: a measurement part configured to measure skin temperatures at a plurality of locations on a face of a user; a calculation part configured to calculate a variation of the skin temperatures at the plurality of locations; and a blood flow assessment part configured to assess a blood flow of the user based on the variation.

### TECHNICAL FIELD

The present invention relates to a blood flow assessment device, a method, and a program.

### BACKGROUND ART

Conventionally, it is known that blood flow is related to skin temperature (see non-patent document 1). For example, in patent document 1, the blood flow rate is estimated based on changes in skin temperature.

### CITATION LIST

### Patent Documents

Patent Document 1: International Publication No. WO2010/143251

### Non-Patent Documents

Non-Patent Document 1: Masami IRIKI, “Body Surface Temperature Physiology,” BME Vol. 3, No. 7, 1989, pp. 9-15

### SUMMARY OF INVENTION

### Technical Problem

However, skin temperature varies with location on the skin. Therefore, according to conventional methods, when the skin temperature is measured at different locations, the estimated blood flow will also differ. As a result of this, there is a possibility that the blood flow cannot be assessed correctly.

Accordingly, an object of the present invention is to improve the accuracy of blood flow assessment.

### Solution to Problem

A blood flow assessment device according to one embodiment of the present invention includes: a measurement part configured to measure skin temperatures at a plurality of locations on a face of a user; a calculation part configured to calculate a variation of the skin temperatures at the plurality of locations; and a blood flow assessment part configured to assess a blood flow of the user based on the variation.

### Advantageous Effects of the Invention

According to the present invention, it is possible to improve the accuracy of blood flow assessment.

### BRIEF DESCRIPTION OF DRAWINGS

FIG. 1 is a diagram that illustrates an overall system structure according to one embodiment of the present invention;

FIG. 2 is a diagram that shows functional blocks of a blood flow assessment device according to one embodiment of the present invention;

FIG. 3 is a flowchart that shows the flow of a blood flow assessment process according to one embodiment of the present invention;

FIG. 4 is a diagram for explaining a questionnaire according to one embodiment of the present invention;

FIG. 5 is a transition diagram of screens displayed on a blood flow assessment device according to one embodiment of the present invention; and

FIG. 6 is a diagram that shows a hardware structure of a blood flow assessment device according to one embodiment of the present invention.

### DESCRIPTION OF EMBODIMENTS

Now, an embodiment of the present invention will be described below with reference to the accompanying drawings. Note that, in the present specification and the accompanying drawings, components having substantially the same functional structure are assigned the same reference numerals, thereby omitting redundant description.

### Description of Terms

“Blood flow” means the flow of blood. Note that this is also referred to as “blood circulation.”

“Skin temperature” refers to the temperature of the surface of the body. Note that this is also referred to as “surface temperature” or the like.

<System Structure>

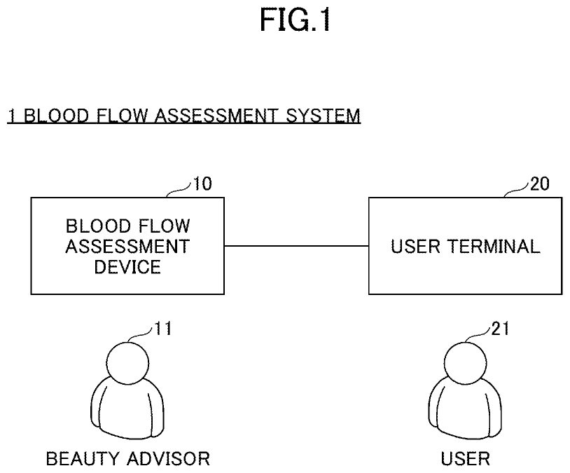

FIG. 1 is a diagram that shows an overall system structure according to one embodiment of the present invention. As shown in FIG. 1, a blood flow assessment system 1 includes a blood flow assessment device 10 and a user terminal 20. The blood flow assessment device 10 can send data to and receive data from the user terminal 20 via any network. Each of these will be described below.

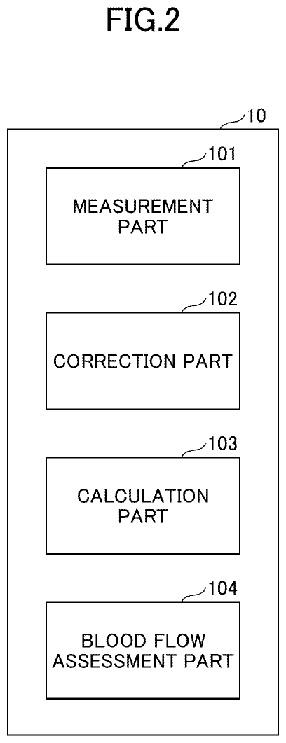

The blood flow assessment device 10 assesses the blood flow of a user 21. To be more specific, the blood flow assessment device 10 measures skin temperature at multiple locations on the face of the user 21. Also, the blood flow assessment device 10 calculates the variation of skin temperature at multiple locations. Also, the blood flow assessment device 10 assesses the blood flow of the user 21 based on the variation. For example, the blood flow assessment device 10 is a smart mirror with a built-in thermography function (to be more specific, a function to analyze the infrared rays emitted from the face of the user 21 and generate an image that graphically represents the thermal distribution). Below, the blood flow assessment device 10 will be described in detail with reference to FIG. 2.

The user terminal 20 is a terminal for acquiring blood flow assessment results. For example, the user terminal 20 is a smartphone or the like.

Note that, although this specification will describe a case in which the blood flow assessment device 10 is one device (for example, a smart mirror or the like), the blood flow assessment device 10 may be composed of a plurality of devices (for example, a thermography camera and a tablet terminal).

For example, the blood flow assessment device 10 is installed in a store that sells cosmetics. The beauty advisor 11 can guide the user 21 to use the blood flow assessment device 10. The user terminal 20 of the user 21 can acquire the blood flow assessment results by e-mail or the like.

<Functional Block of Blood Flow Assessment Device>

FIG. 2 is a diagram that shows functional blocks of the blood flow assessment device 10 according to one embodiment of the present invention. The blood flow assessment device 10 may include a measurement part 101, a correction part 102, a calculation part 103, and a blood flow assessment part 104. Also, the blood flow assessment device 10 can function as the measurement part 101, the correction part 102, the calculation part 103, and the blood flow assessment part 104 by executing programs.

The measurement part 101 measures the skin temperature at multiple locations on the face of the user 21 (for example, the forehead, the right cheek, the left cheek, the nose, the chin, etc.).

The measurement part 101 can measure the skin temperature at multiple locations on the face of the user 21 after a period of time corresponding to a condition that is related to the user 21 and that directly or indirectly influences the skin temperature at multiple locations on the face of the user 21 elapses. For example, if the user 21 wears a mask up until blood flow assessment, the measurement part 101 can measure the skin temperature after a period of time corresponding to the time the user 21 has worn the mask elapses (for example, the longer the mask has been worn, the longer the period of time). For example, the measurement part 101 can measure the skin temperature after a period of time corresponding to the heart rate of the user 21 elapses (for example, the higher the heart rate, the longer the period of time).

The correction part 102 corrects the skin temperatures measured by the measurement part 101 according to a condition that is related to the user 21 and that directly or indirectly influences the skin temperature at multiple locations on the face of the user 21. For example, if the user 21 wears a mask up until blood flow assessment, the correction part 102 can correct the skin temperatures according to how long the user 21 has worn the mask (for example, the correction part 102 lowers the skin temperature more the longer the user 21 has worn the mask). For example, the correction part 102 can correct the skin temperatures according to the heart rate of the user 21 (for example, the correction part 102 lowers the skin temperature as the heart rate of the user 21 increases).

The calculation part 103 calculates the variation of skin temperature at multiple locations on the face of the user 21. To be more specific, the calculation part 103 calculates the variation of skin temperatures corrected by the correction part 102 (when skin temperature is subject to correction), or calculates the variation of skin temperatures measured by the measurement part 101 (when skin temperature is not subject to correction).

<Calculation of Variation of Skin Temperature>

Here, how to calculate the variation of skin temperature will be described in detail below. Note that “variation” may refer to any value that indicates variation, such as the magnitude of variation, the bias of variation, and so forth. Now, <Calculation of the magnitude of variation in skin temperature> and <Calculation of the bias of variation in skin temperature> will be described below.

<Calculation of the Magnitude of Variation in Skin Temperature>

For example, the calculation part 103 can calculate the interquartile range of skin temperature at multiple locations on the face of the user 21, and use this interquartile range as a score of variation of skin temperature (in other words, the greater the variation of skin temperature, the higher the score).

For example, the calculation part 103 can calculate the average deviation of skin temperature at multiple locations on the face of the user 21, and use that average deviation as a score of variation of skin temperature (in other words, the greater the variation of skin temperature, the higher the score).

For example, the calculation part 103 can calculate the variance of skin temperature at multiple locations on the face of the user 21, and use this variance as a score of variation of skin temperature (in other words, the greater the variation of skin temperature, the higher the score).

For example, the calculation part 103 can calculate the standard deviation of skin temperature at multiple locations on the face of the user 21, and use this standard deviation as a score of variation of skin temperature (in other words, the greater the variation of skin temperature, the higher the score).

For example, the calculation part 103 can calculate the coefficient of variation of skin temperature at multiple locations on the face of the user 21, and use this coefficient of variation as a score of variation of skin temperature (in other words, the greater the variation of skin temperature, the higher the score).

For example, the calculation part 103 can calculate the sum of the absolute values of skin temperature differences between respective locations, and use this sum of absolute values as a score of variation of skin temperature (in other words, the greater the variation of skin temperature, the higher the score). For example, the calculation part 103 can use the sum of the absolute values of the differences between a certain location (for example, a location where the temperature is stable, such as the forehead) and other locations (for example, locations where the temperature is not stable, such as the right cheek, the left cheek, the nose, the chin, and so forth) as a score of variation of skin temperature (for example, “|forehead skin temperature−right cheek skin temperature|+|forehead skin temperature−left cheek skin temperature|+|forehead skin temperature−nose skin temperature|+|forehead skin temperature−chin skin temperature|”).

For example, the calculation part 103 can calculate the sum of the differences in skin temperature between locations, and use this sum as a score of variation of skin temperature. For example, the calculation part 103 can determine the sum of the differences between a location (for example, the location with the highest temperature) and other locations as a score of variation of skin temperature.

For example, the calculation part 103 can calculate the difference between the skin temperature at the location of the highest temperature and the skin temperature at the location of the lowest temperature, and use this difference as a score of variation of skin temperature.

<Calculation of the Bias in Variation of Skin Temperature>

For example, the calculation part 103 can calculate the bias of skin temperature at multiple locations on the face of the user 21, and use this bias as a score of variation of skin temperature.

The blood flow assessment part 104 assesses the blood flow of the user 21 (for example, a score to indicate blood flow (the volume of blood flow, etc.) based on the variation calculated in the calculation part 103 (for example, a score of skin temperature variation). Note that the blood flow assessment part 104 may display the assessment result on the display device of the blood flow assessment device 10, or transmit the assessment result to the user terminal 20 by e-mail or the like.

Conventionally, it is known that skin temperature is low when the blood flow is poor, and skin temperature is high when the blood flow is good. In other words, it is likely that poor blood flow causes areas with low skin temperature, resulting in areas with high skin temperature and areas with low skin temperature (that is, variation in skin temperature arises). Therefore, the blood flow assessment part 104 can assess that the blood flow of the user 21 is better when the variation is smaller. Also, the blood flow assessment part 104 can assess that the blood flow of the user 21 is poorer when the variation is larger.

In addition, the blood flow assessment part 104 can assess the blood flow of the user 21 based on a condition that is related to the user 21 and that directly or indirectly influences the skin temperature at multiple locations on the face of the user 21. For example, the blood flow assessment part 104 can assess the blood flow of the user 21 further based on the score on a questionnaire. The score on a questionnaire is determined based on a questionnaire (to be more specific, questions regarding conditions that are related to the user 21 and that directly or indirectly influence the skin temperature at multiple locations on the face of the user 21, including, for example, “I feel cold with the air conditioner,” “My cheeks are red,” “I got sunburned recently,” etc.).

Here, the score to indicate blood flow will be described. The blood flow score may be based solely on the score of variation of skin temperature, or may be based on the sum of the score of variation of skin temperature and the score on a questionnaire. For example, the blood flow assessment part 104 can give a higher blood flow score for better blood flow, and give a lower blood flow score for poorer blood flow. For example, the blood flow assessment part 104 can use variables to broaden the range of distribution of scores indicative of blood flow. For example, “100−variable×(score of variation in skin temperature+score on a questionnaire)” can be used as a score that indicates blood flow.

<Processing Method>

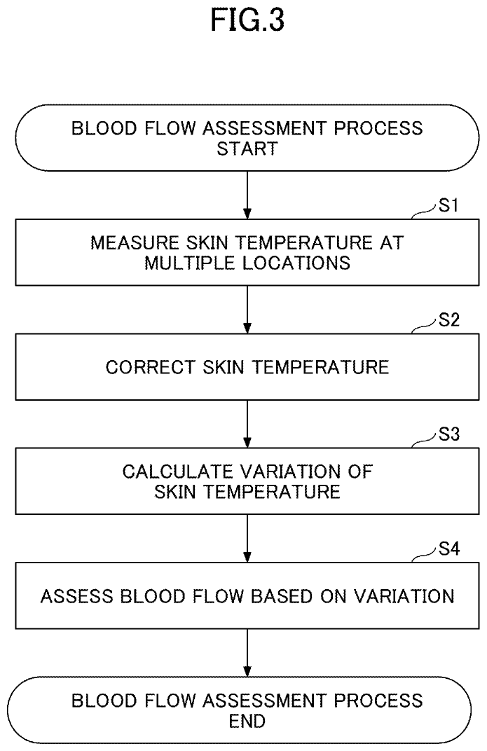

FIG. 3 is a flowchart that shows the flow of a blood flow assessment process according to one embodiment of the present invention.

In step 1 (S1), the measurement part 101 measures the skin temperature at multiple locations on the face of the user 21.

Note that the measurement part 101 can measure the skin temperature at multiple locations on the face of the user 21 after a period of time corresponding to a condition that is related to the user 21 and that directly or indirectly influences the skin temperature at multiple locations on the face of the user 21 elapses.

In step 2 (S2), the correction part 102 corrects the skin temperatures measured in S1 according to the condition of the user 21 that directly or indirectly influences the skin temperature at multiple locations on the face of the user 21.

Note that S2 may be omitted.

In step 3 (S3), the calculation part 103 calculates the variation of skin temperature at multiple locations on the face of the user 21. To be more specific, the calculation part 103 calculates the variation of skin temperatures corrected in S2 (when S2 is executed), or calculate the variation of skin temperatures acquired in S1 (when S2 is not executed).

In step 4 (S4), the blood flow assessment part 104 assesses the blood flow of the user 21 based on the variation calculated in S3.

Note that the blood flow assessment part 104 may assess the blood flow of the user 21 further based on a condition that is related to the user 21 and that directly or indirectly influences the skin temperature at multiple locations on the face of the user 21.

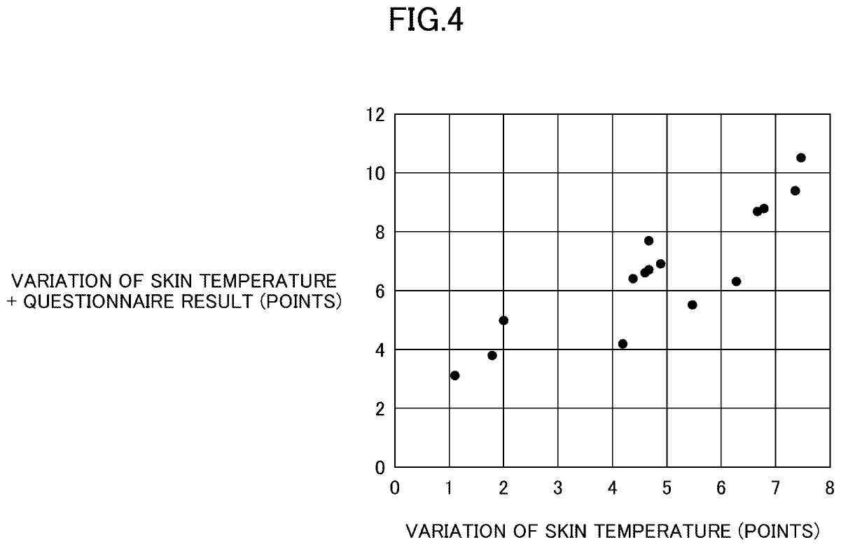

FIG. 4 is a diagram for explaining a questionnaire according to one embodiment of the present invention. To be more specific, FIG. 4 shows the scores of skin temperature variation and the sums of the scores of skin temperature variation and the scores on a questionnaire, for each subject. The horizontal axis in FIG. 4 is the score of skin temperature variation (ranging from 0 points to 8 points), and the vertical axis in FIG. 4 is the sum (ranging from 0 points to 12 points) of the score of skin temperature variation and the score on a questionnaire. In FIG. 4, the content of the questionnaire include “I feel cold with the air conditioner (the colder the subject feels with the air conditioner, the higher the score),” “My cheeks are red (the redder the subject's cheeks are, the higher the score),” and “I recently got sunburned (the more recently the subject got sunburned, the higher the score).”

As shown in FIG. 4, as the score of skin temperature variation (the score on the horizontal axis) increases (that is, poorer blood flow), the sum of the score of skin temperature variation and the score on the questionnaire (the score on the vertical axis) increases (that is, poorer blood flow). From this, it can be seen that the content of the questionnaire in FIG. 4 indicates conditions that are related to the user 21 and that directly or indirectly influence the skin temperature at multiple locations on the face of the user 21.

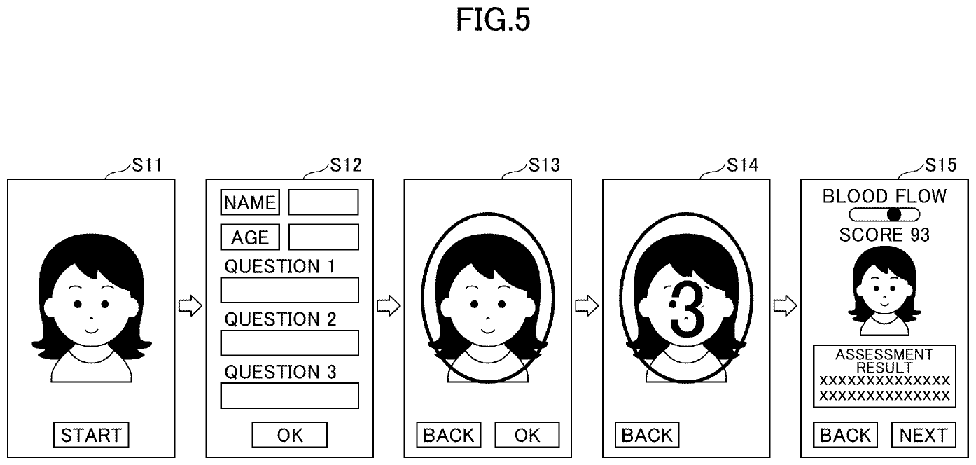

FIG. 5 is a diagram that shows the transition of screens displayed on the blood flow assessment device 10 according to one embodiment of the present invention.

In step 11 (S11), a screen for starting blood flow assessment is displayed. When the beauty advisor 11 or the user 21 selects the start button on the screen of S11, the process proceeds to step 12.

In step 12 (S12), a screen for answering a questionnaire (to be more specific, questions regarding conditions that are related to the user 21 and that directly or indirectly influence the skin temperature at multiple locations on the face of the user 21) is displayed. When the beauty advisor 11 or the user 21 answers the questions on the screen of S12 and selects the OK button, the process proceeds to step 13.

In step 13 (S13), a screen to guide the face of the user 21 to be placed inside a frame is displayed. When the user 21 adjusts his/her face so that it fits in the frame of the screen of S13 and the beauty advisor 11 or the user 21 selects the OK button, the process proceeds to step 14.

In step 14 (S14), a screen to show that the blood flow assessment is in progress is displayed. When the blood flow assessment is finished, the process moves on to step 15.

In step 15 (S15), a screen to show the result of blood flow assessment is displayed. For example, the result of blood flow assessment includes a score that indicates blood flow.

<Effect>

Thus, according to the present invention, blood flow is assessed based on the variation of skin temperature at multiple locations on the face, so that the accuracy of blood flow assessment can be improved.

<Hardware Structure>

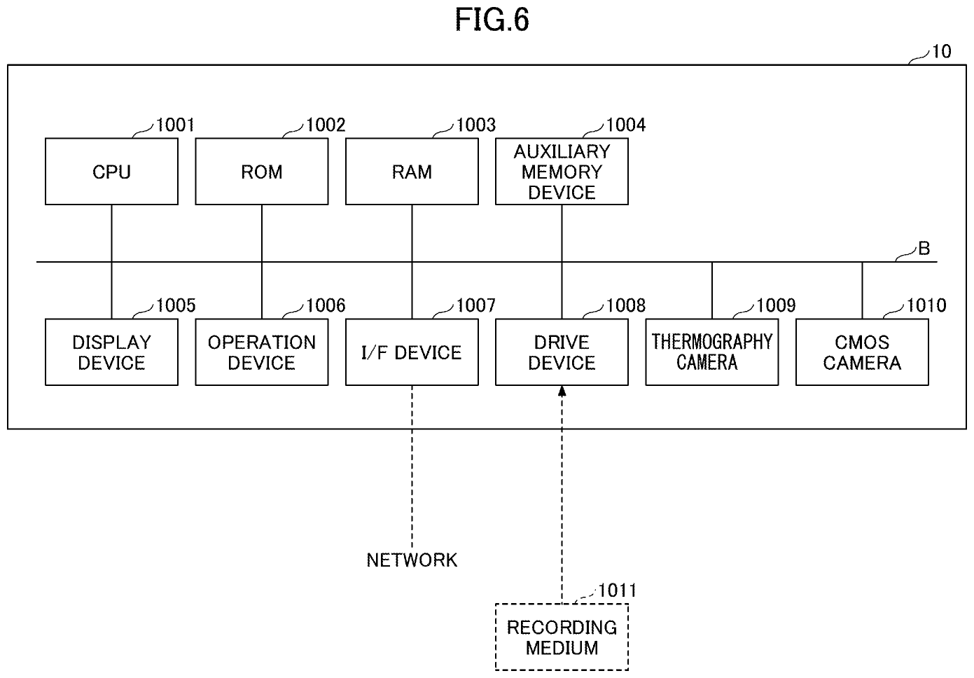

FIG. 6 is a hardware structure diagram of the blood flow assessment device 10 according to one embodiment of the present invention.

The blood flow assessment device 10 has a thermography camera 1009 and a CMOS camera 1010. The thermography camera 1009 measures the skin temperature of the user 21. The CMOS camera 1010 photographs the user 21.

The blood flow assessment device 10 has a central processing unit (CPU) 1001, a read only memory (ROM) 1002, and a random access memory (RAM) 1003. The CPU 1001, the ROM 1002, and the RAM 1003 constitute what is known as a computer.

Also, the blood flow assessment device 10 can have an auxiliary memory device 1004, a display device 1005, an operation device 1006, an interface (I/F) device 1007, and a drive device 1008.

Note that each piece of hardware of the blood flow assessment device 10 is interconnected via a bus B.

The CPU 1001 is an arithmetic device that executes various programs installed in the auxiliary memory device 1004.

The ROM 1002 is a non-volatile memory. The ROM 1002 functions as a main memory device that stores various programs, data, and so forth, that are necessary for the CPU 1001 to execute various programs installed in the auxiliary memory device 1004. To be more specific, the ROM 1002 functions as a main memory device that stores boot programs such as a basic input/output system (BIOS) or an extensible firmware interface (EFI).

The RAM 1003 is a volatile memory such as a dynamic random access memory (DRAM) or a static random access memory (SRAM). The RAM 1003 functions as a main memory device that provides a working area that is developed when various programs installed in the auxiliary memory device 1004 are executed by the CPU 1001.

The auxiliary memory device 1004 is an auxiliary memory device that stores various programs and information for use when various programs are executed.

The display device 1005 displays the internal state of the blood flow assessment device 10 and the like.

The operation device 1006 is an input device through which a person operating the blood flow assessment device 10 inputs various instructions to the blood flow assessment device 10.

The I/F device 1007 is a communication device for connecting with a network and communicating with other devices.

The drive device 1008 is a device into which the recording medium 1011 is placed. The recording medium 1011 here may refer to a medium that records information optically, electrically or magnetically, such as a CD-ROM, a flexible disk, a magneto-optical disk, and so forth. Also, the recording medium 1011 may refer to a semiconductor memory that electrically records information such as an EPROM (Erasable Programmable Read Only Memory), a flash memory, or the like.

Note that various programs to be installed in the auxiliary memory device 1004 are installed by, for example, setting the distributed recording medium 1011 to the drive device 1008 and reading various programs recorded on the recording medium 1011 through the drive device 1008. Alternatively, various programs installed in the auxiliary memory device 1004 may be installed by being downloaded from the network via the I/F device 1007.

Although an embodiment of the present invention has been described in detail above, the present invention is not limited to the specific embodiment described above, and various modifications and changes can be made without departing from the scope of the present invention recited in the claims.

This international application is based on and claims priority to Japanese Patent Application No. 2021-001054, filed on Jan. 6, 2021, the entire contents of which are herein incorporated by reference.

### REFERENCE SIGNS LIST

1 blood flow assessment system
10 blood flow assessment device
11 beauty advisor
20 user terminal
21 user
101 measurement part
102 correction part
103 calculation part
104 blood flow assessment part
1001 CPU
1002 ROM
1003 RAM
1004 auxiliary memory device
1005 display device
1006 operation device
1007 I/F device
1008 drive device
1009 thermography camera
1010 CMOS camera
1011 recording medium

1. A blood flow assessment device comprising:
a measurement part configured to measure skin temperatures at a plurality of locations on a face of a user;
a calculation part configured to calculate a variation of the skin temperatures at the plurality of locations; and
a blood flow assessment part configured to assess a blood flow of the user based on the variation.

2. The blood flow assessment device according to claim 1, wherein the blood flow assessment part assesses the blood flow of the user further based on a condition that is related to the user and that directly or indirectly influences the skin temperatures at the plurality of locations on the face of the user.

3. The blood flow assessment device according to claim 1, further comprising a correction part configured to correct the skin temperatures according to a condition that is related to the user and that directly or indirectly influences the skin temperatures at the plurality of locations on the face of the user,
wherein the calculation part calculates a variation of the corrected skin temperatures.

4. The blood flow assessment device according to claim 1, wherein the measurement part measures the skin temperatures at the plurality of locations on the face of the user after a period of time corresponding to a condition that is related to the user and that directly or indirectly influences the skin temperatures at the plurality of locations on the face of the user elapses.

5. The blood flow assessment device according to claim 1, wherein the variation is a magnitude of the variation.

6. A method comprising:
measuring skin temperatures at a plurality of locations on a face of a user;
calculating a variation of the skin temperatures at the plurality of locations; and
assessing a blood flow of the user based on the variation.

7. A non-transitory computer-readable recording medium storing a program that causes a blood flow assessment device to function as:
a measurement part configured to measure skin temperatures at a plurality of locations on a face of a user;
a calculation part configured to calculate a variation of the skin temperatures at the plurality of locations; and
a blood flow assessment part configured to assess a blood flow of the user based on the variation.
